(19) United States
(12) Patent Application Publication
(10) Pub. No.: US 2010/0145398 A1
(43) Pub. Date: Jun. 10, 2010

(54) Cancer Treatment Equipment

(76) Inventors: Fusheng Li, Qingdao City (CN); An Li, Qingdao City (CN); Jing Du, Qingdao City (CN); Xiaomei Yang, Qingdao City (CN)

Correspondence Address: HAYES, SOLOWAY P.C., 175 CANAL STREET, MANCHESTER, NH, 03101, US

(21) Appl. No.: 12/514,673

(22) PCT Filed: Nov. 5, 2007
(86) PCT No.: PCT/CN07/03135

§ 371 Date: Dec. 1, 2009

(30) Foreign Application Priority Data
Nov. 13, 2006 (CN) 200610069987.8

(51) Int. Cl. A61N 1/00; A61N 1/30

(52) U.S. Cl. 607/3; 604/20

(57) ABSTRACT

A cancer treatment equipment includes a case (7), a squeeze-type friction head (1) connected to direct current, a stiction friction head (2) of low-middle frequency current, a friction head (3) with magnetic sheet, a medicated gauze pad fixation clamp (4), a medicated gauze pad (5) and a treatment device of RF, microwave or ultrasonic. The squeeze-type friction head (1) connected to direct current, the stiction friction head (2) of low-middle frequency current and the friction head (3) with magnetic sheet are fixed in the case (7) in parallel. The medicated gauze pad (5) is fixed on the medicated gauze pad fixation clamp (4) on the lower part of the case (7).

### TECHNICAL FIELD

This invention belongs to the scope of medical devices, and more in detail, it belongs to a medical device used for treatment of cancer through combination of massage with drugs, physical therapy, local injection and administration.

### BACKGROUND ART

Different from common diseases, major diseases such as cardio-cerebrovascular diseases, cancers, uremia, diabetes, tuberculosis, hepatitis and AIDS impose huge risks to human life and the increasing trend in their morbidity and mortality can't be reduced or they can't be cured through the existing medical technologies. The modern medicine aims at variances at the same level. A clear diagnosis on the location, situation and physicochemical properties of major diseases can be made in the clinic medicine as to identify the diseases' features according to part of causes in their structures. However, there is lack of treatment in functions. As reported from the Reference News on Aug. 3, 2004, the white paper of Ministry of Health, Labor and Welfare, Japan indicated that the cure rate of disease still remains at 20% without any improvement although a large amount of human and financial resources are input. The United States hasn't made any substantial improvement in the research on cancer since the 1970s, which indicates that we have one-sided understanding of the law of generation and development of cancers and major diseases; otherwise there would not be a universal situation of difficulty in cure. The human body is a unity of interactive multi-level structures and the life of human body is a high-level form of movement in the nature. Major diseases can threaten human life in that they are integrated diseases in high-level forms of movement with interactive multi-level structures as they are and need comprehensive treatment in high-level forms of movement with interactive multi-level structures. Due to lack of overall understanding of the world, the modern medicine understands diseases with one sidedness in low-level local analysis instead of the holistic human body, which leads to directional mistakes in the fundamental medicine as not to link the clinical treatment with the cure goals. It's just a partial efficacy-limited treatment and impossible to achieve the goal of curing cancers. The only resolution to this problem is to repeat an overall understanding of the law of generation and development of diseases and propose innovative fundamental medicine theory and clinical treatment technologies for major diseases.

The modern medicine, when used for treatment of cancers, rely much more on drugs, surgeries and radiotherapies that cure outward symptoms rather than root causes and can't make the body recovered, neglecting the primary principles of science—the overall interaction of human evolution and natural selection and resulting in being unclear of pathogenesis so far and finding no basic treatment method and inability to cure both outward symptoms and root causes. The treatments such as drugs, surgeries and radiotherapies all aim to the symptoms and lack of curing root causes, can't stop the upward trend in mortality and morbidity.

1. Of the existing technologies, surgeries, radiotherapies and chemotherapies are the main treatments for cancers. Their curative effects are limited when pathological changes equally occur on the holistic in the two factors of treatment structure and functions. It can't reverse the direction and trend of development of cancer, not cure the intermediate and advanced cancers or not significantly reduce mortalities; even these cancers are considered as “incurable”.

2. The primary treatment of independent physical therapies and chemical therapies, great in side effects and low in effect.

3. In function recovery, lack of specific location therapy.

4. In function recovery, the devices using the existing technologies for treating visceral diseases include: ZL951121065, a massage device: ZL981103057, which is a drug massage device through mechanical actions and lacks of appropriate combination of sound, light, electricity, magnetism and heat, so the physical therapy is limited. Due to lack of combination with chemical therapy of local injection and other local administrations, it can't form an advanced treatment technology to meet the need of overall and deep cancer treatment.

### BRIEF SUMMARY OF THE INVENTION

The purpose of this invention is to make a functional recovery of pathogenic locations, prevent formation of cancers and conduct clinical treatment and recovery, using the advanced treatment technology with combination of drug massage, local injection, MHF electrotherapy, ultrasound and microwave according to the treatment principles and methods of major diseases and the basis that the holistic interaction of decline in functions and structure injuries of human body is the overall cause of cancer formation. Compared to the partial cause treatment such as surgeries, radiotherapies and local chemotherapies, the interactive promotion of symmetrical equivalence constitutes the method for overall cause treatment and therefore it can produce an overall effect and achieve the goal of curing diseases. In order to fully publicize the invention, the relevant principles and technologies of cancer treatment will be explained in detail.

### (I) Rules of Generation, Development and Improvement of Major Diseases

The interdependent and harmonious movement of human and the nature forms the basis for existence and development of life, and similarly, the decline in the function balancing Yin and Yang for metabolism and interaction of inner structural causes and outward functional causes is the basis for generation of diseases. The contention of structural injuries and anti-injuries is the driving force of development on this basis. The essences of major diseases result from interaction of inner and outward causes of the contradiction theory of Philosophy and have universal significance. Its manifestation—symptom—is one of diversity in structures and functions of scientific systematic theory and varies from person to person. Philosophical contradiction theory and scientific system theory, inner structures and outward functions, individuals and holistic, universal essences and specific representations, self-development and overall development are the two sides of one problem, which are interdependent and interactive. The cause for cancer is a holistic composed of inner and outward reasons, meaning not only specific representations of endogenous structural factors, but also the deeply essences of physical energy decline in exogenous functions and general factors of modern unhealthy lifestyle. Cancers should be cured targeting at endogenous causes, exogenous causes, and holistic causes both endogenous and exogenous. The recovery function can balance Yin and Yang and regulate the development direction and trend of diseases and specific therapies and scales. The contrast treatments of anti-injuries struggle decide the development speed and situation of diseases. The surgeries, radiotherapies and chemotherapies of the modem medicine are the ways to kill cancer cells, targeting at the endogenous structures rather than exogenous causes for generation of cancer cells, they are maintaining and remedial semi-treatments, not targeting at the holistic causes and can't product an overall causal changes. The real medical researches focus on the holistic causes, causality and human physiology and pathology as well as rather simple treatments.

The motherland medicine believes that the accordance and harmony should be kept between human and the heaven, to support healthy trends and get rid of bad energy, to address both outward symptoms and root causes and to balance Yin and Yang. Such overall treatment principles based on the ancient philosophy have been proven correct in accordance with the natural law by experiencing several thousand-year practices, which can develop by itself and also together with the Western medicine. However, this combination can be really achieved only through their counterparts. The Chinese medicine should be based on the modern philosophy and have the expertise in restoration of functions, taking the modern sciences as the base and focusing on combination with the anti-injury Western medicine. The specific technology, if going to develop, should follow the general law of natural development, because the philosophy studies the general laws of development and improvement and its own starting point is unitary in opposite and unity. Different opinions on such unitary conception will result in different treatment directions and outcomes. All the diagnosis, prevention and treatment of the clinical medicine are based on the belief of philosophic causality, partial causal outcomes only from the partial treatments while holistic from holistic. The cause of major diseases such as cancers lies in reduction of holistic functions and local injuries and the significant effect only comes from holistic treatment with counterpart in restoration of functions and anti-injuries.

In terms of understanding the rule on natural development, the modern natural philosophy has three views on the relationship of struggle and unity of things, whether the unity exists in the quality changes of things' development and what's the motivation of things' development: one is that the positions of struggle and unity can be transformed mutually, sometimes the struggle plays a decisive role and sometimes the unity plays; another is that in the changes to things' quality, the struggle is the motivation of development as well as the decisive factor while the unity doesn't play a role; the third view is that the decisive role of the unity or the struggle, as the motivation of things' development, is a mutual combination to build into driving force and decisive factors rather than a matter of “sometimes”. See ZHE HAI TAN HANG, Bo Xiangji, Xiyuan Press, 1^{st} Edition, March 2000 (Page 193-194). The first and the second views are related to quantity changes and partial actions, coming from the chapter 2 “Introduction of Diseases” of a higher education textbook of the modern medicine Pathophysiology, 6^{th} Edition. Because of the relationship of opposite and unity between anti-injury struggle and function restoration unity, physical and chemical aspects, it may play a decisive role only in the quantity change and at a low level through self-development according to the partial rules in a way of one into two, and also it may play a decisive role in the quality and at a high level according the holistic rules in a way of two in one. “Sometimes”, the mutual transformation of two factors with one in the dominant position is a change in the quantity of things' sizes and weights in the process of development while the interdependent quality of two aspects of the thing doesn't happen to any change. The quality change of thing needs “simultaneous” actions from the two factors. Lack of identity can not combine into a whole and cause changes to the quality of things, which belongs to a partial therapy and apply to common disease due to partial causes. The principle and method for treatment of major diseases adopts the third views on the holistic quality changes in a way of two in one, because only the two counterparts of self-development play simultaneously, the high-level holistic quality change can occur. Cancer patients have suffered from structural lesion and functional reduction, physical and chemical form of pathological changes in a way of holistic interaction. So in the treatment of cancer, combining the anti-injury struggle treatment such as surgeries, radio- and chemotherapies to kill cancer cells and the unitary treatment of function restoration to eliminate cancer cells, physical therapy and chemotherapy, is a co-action to mutually improve the holistic quality, which decides the development and improvement of cancers instead of competition of anti-injury strengths. The anti-injury treatment is one component of the holistic therapy, just play a partial role and can't mutually improve and combine with function restoration in counterpart. It would be restrained by reduction of functions and be limited in efficacy. This is why common diseases can be cured but the long-term weak patients with cardio-cerebrovascular diseases and cancers can't be cured due to their recurrence. Patients with major diseases have the death outcome due to a holistic effect of functional reduction and structural lesion. The restoration function will not produce such interactive lethal holistic effect, patients, although seriously ill, will not die. As Xu Lingdai, a medical scientist in the Qing Dynasty said in his book Medicine Origins: “if pneuma is not damaged, a disease, although serious, will not cause death. If pneuma is damaged, a disease, although not serious, will cause death”. At the same time, employ a holistic therapy combining functional restoration and anti-injury treatment will release patients from death threat.

### (II) Principles of Major Diseases

The existing technological theories (including traditional medicine and modern medicine) believe that the structural lesion and the functional decline can be classified which is dominant and which is subordinate, so curing well the dominant factor and assisting in curing the subordinate aspect, the disease may be cured. Such treat protocol taking partial factors as holistic is right to cure common diseases with predominance in treatment of partial factors; however, it can't work out in curing major diseases evolving from common diseases due to changes in their qualities. This is in that the subordinate factors have significant changes and become the interdependent counterparts. It's the holistic factor that plays a decisive role instead of partial factors, which is the essence of major diseases. Currently, the acute stage of major diseases is subject to anti-injury treatment and the stable stage is subject to functional restoration, such separation treatment is partial and limited in its efficacy. Because no matter how the pathological change is, it is an outcome of their interaction. Major diseases have been experiencing from movement at any time as a whole, without one dominant and detachable process. Major diseases such as cancers, cardio- and cerebrovascular disease, diabetes, uremia, liver disease, leukemia and AIDS are holistic diseases of multi-level interactive structures and high-level movement. According to the principle of holistic therapy combining functional restoration and anti-injury counterpart in a way of supporting healthy trends and getting rid of bad energy, these diseases can be cured. It's essentially different from the modem medicine only focusing on anti-injury medical theory and employing primary technology only to produce partial efficacy in treatment of major diseases.

With regard to prevention and treatment, the cause and location of disease is more important than its symptom. For the holistic treatment of cancer, only aiming at the generation site the treatment goal can be achieved.

(1) Principle of treatment: major disease can be holistically treated with a high-level aiming technology through identifying the scope of pathological change in the whole function decline interdependent to the last level at which the local scope of pathological changes is diagnosed.

(2) Prevention of major diseases: because causes are a whole effect of structural damage and functional decline, a high-level movement both physical and chemical, and therefore, only use primary physical or chemical forms of treatment technology to prevent functions from decline of pathological changes, major diseases can't form from mutual constraints of factors. In terms of diabetes, liver disease, uremia, cardiovascular and cerebrovascular diseases, cancer and AIDS, there is 100% existence of blood circulation, microcirculation, metabolism decreasing barrier around structural lesions, which is the common cause for major diseases as well as the reason for not cure such diseases. Always treat these sites in a way of balance of functional restoration; such barriers can be eliminated so that major diseases can be prevented. Together with anti-injury treatment, it may achieve the purpose of healing. Additionally, cardio- and cerebrovascular diseases and cancers are chronic with long-term decline in the body's own functions, while infectious diseases are acute due to increase of pathogenic conditions, so it should distinguish prevention and treatment for different diseases.

(3) Incidence sites and positioned treatment of major diseases

Human diseases can be leveled and need to treat at the deepest level according to the levels of pathological change, if not, the effect would be limited, resulting in misdiagnosis and iatrogenic diseases, which especially manifest in the treatment of major diseases. The core of treatment depth is how to restore the level of function. For example, repairing damage of cancer cells not only focuses on structural damage of genes, but also on restoration of the peripheral metabolic functions of genetic structure. At present, lack of study on gene structure and peripheral functions as well as lack of function restoration results in efficacy-limited gene therapy. The existing technology lacks deeply understanding how human health is affected, it is difficult to find organs or tissues corresponding to structural treatment and not to find pathogenic sites to be functionally restored. In terms of such issue, the whole treatment theory has not made a clear research or answer, resulting in a lack of functional treatment to the local structure around the tumor, which is the root cause for cancers can not be cured. Whether modem medicine or traditional medicine, both are of the view that the tumor is a local performance of systemic diseases, required local and systemic treatment, but patients don't happen to the need of first aid for body functions. Changes in body functions and pathogenesis of the local structure are a secondary indirect role and there is no overall constraint relationship of their counterparts. It's the pathogenesis occurring to the holistic function of the local structure around the cancer focus that leads to lack of specific treatment of functional restoration for cancers, so it should be at the level of overall pathogenesis of the cancer structure.

Individual living activities and pathogenesis of human body are holistic in interaction between the low-level local structure and the high-level overall function. When the local structure happens to pathogenesis, the local functions at the same level no longer exist; it is the interaction between the high-level overall function including local functions and local structure with pathological changes. When both lesions have a reverse change due to mutual constraints, it will enter the process of interaction between the overall structure change and one higher level of the overall function. Molecular structures and cell function, cell structures and tissue functions, tissue structures and organ functions, organ structures and system functions, system structures and functions of human body, structure of human body and function of human society, human social structure and system functions related to the natural environment, the relevant natural system structure and function of the Earth's environment, the overall structure of the Earth and the universe's system functions are of interdependent and harmonious movements.

Modern medicine pay more attention to Pathology, positioning diseases at the levels of organs, tissues, cells and molecules and considering them as pathological changes at the same level that the essence of the disease is “local positioned organic pathogenesis”. It lacks of knowledge about “the overall positioned function change” of diseases, and can not recognize the intrinsic properties and can observe some factors of disease only. Due to the lack of the overall concept of interdependence of the local structure and overall function, simply from the anatomical structure to understand the unity at the molecular and cellular level is far from formation of the theoretical system reflecting movement and development rules at molecular, cellular and overall levels. Local structure pathogenesis at low level of major diseases due to internal causes, and the overall function pathogenesis at high level due to external causes, interdependently constitute a unified whole, and their locations are in the adjacent area between the two levels. Such as the specific location of cancer, the early stage of lesion indicates between the local structure at the cellular level and the overall function at the tissue level; the intermediate stage of lesion refers to that between the local structure at the tissue level and the overall function at the organic level; the late stage of lesion refers to that between the local structure at the organic level and the overall function at the systematic level; the first-aid refers to that between the system-level structure and the function of human body. Function refers to the microcirculation and metabolic functions of oxygen and nutrients supply and absorbance. Modern medicine has more levels of awareness on the human body structure, but inadequate on the function level of the human body, with lack of specific targeted restore function for cancer treatment. The overall treatment refers to that prevents major diseases from deterioration and condition worsened, so that one structure or function can be controlled and overall mutually constrained lesions can be eliminated to make it transformed into common diseases unable to threaten human life diseases, and continue to treatment to recover. To this end a major disease mapping and a positioning treatment plan are shown as follows: As can be seen from the drawings that the development of disease is from a low level to a high level, threatening existence of one upper level; the development of treatment is from the high level to the low level, eradicating diseases. The local organic structure is indissociable to the overall systematic function, the existing technologies for restoration aim at the whole human body, rather than the overall function of focus that can't produce an overall treatment effect. As shown in this graph, when the tissue structure happens to damage, it is necessary to restore the functions of outer organs at the same time to conduct anti-injury treatment. For organic structure damage, it is necessary to restore the function of external-cause system at the same time to perform anti-injury treatment. The function restoration should be implemented with targeted therapy. From the graph on the relationship between human and the nature, it can be seen that the existence of human society is interdependent and harmonious with the improvement of the natural environment, if both sides lack of harmonious movement, a mutual constraints will be a threat to their existence as one disappears, also the other would not exist. At present, the function of the natural environment interdependent to the human society, although a part of the earth function, will develop into a whole global village where human and the nature is harmonious. Therefore, only improving the function of the natural environment and simultaneously improving existence and development of the mankind itself, harmonious in the movement with each other, the human development come can true. Lack of improvement in the natural environment will be constraints of human existence and development. Through the disease picture, you can reveal the relationship of human body's overall function and local structural lesions where the local endogenous structure lesions have their own independent identity and the overall exogenous function pathogenesis also has its own independent identity and location. Their difference can be shown objectively, so do their links, which can lead to the establishment of a new Pathological theory.

### (III) Treatment Technologies for Major Diseases

Man and the nature are interdependent and harmonious in their movement as a whole, life is a harmonious movement, only the harmonious movement human life can be lasting healthy. Human life movement, a form of high-level movement, is the unity of mechanical, physical, chemical forms of natural movements of mutual harmony. The indivisible, detachable mechanical, physical and chemical movements are a form of the primary activities. Existing technology to the human body and disease in the form of high-level campaign comes down to mechanical, physical, chemical forms of low-level movement, and movement of low-level to explain the law of the human body and disease, resulting in the structure and function, the gap between physics and chemistry to separate improve the recovery, the role of anti-injury, and not rely on the combination of the factors the overall high-level treatment, but mainly rely on a single factor in the primary treatment technology, which can prevent serious diseases, cure common diseases, but the overall nature of the treatment major diseases, can not have a significant effect. Cancer patients because of physical, chemical factors are a disease, the integrity of each other constraints, a single chemotherapy treatment, by the symmetry constraints of physical disease, side effects appear low efficacy results, the current antibiotics, chemical treatment as a whole principle of specificity in the treatment of helplessness and embarrassment. Similarly, the present is surgery, radiation therapy physical therapy in the treatment of cancer partial dominant, are on an equal footing, can not be replaced essential anti-cancer chemotherapy does not appear in the local treatment, in which knowledge can not afford a direct role in supporting the body treatment there are some minor treatment. Therefore, anti-injury and recovery functions shall use the function of physical and chemical properties at different levels and equivalently and symmetrically incorporated into a senior treatment as a whole to increase efforts to control the changes in the level of severity, and also recovery factors that are of universal characteristics, treatment is required to be relatively simple. The schematic diagram of the overall high-level treatment is as follows:

In the treatment against cancers, chemical drugs are used for anti-injury treatment and administrated in the local structural damage, not use systemic drug delivery. The drug therapy to restore function is localized around the structural damage with decline in the overall function at the previous level. As can be seen from the chart, in various forms of physical therapies are related to the energy conversion and mechanical movement between mechanical mechanics are sound, light, electricity, magnetic basis of a variety of physical therapy. The mechanics forms the foundation of sound, light, electrical, magnetic and other physical treatments. The physical treatment without mechanical action is incomplete, which can not be combined into a high-level treatment with the chemical treatment. To improve the action of treatment depends not only on the quality and quantity of treatment factors and but also on the combination of them, i.e. the treatment structure. The treatment with chemical drugs alone is a primary technology for pathogenesis resulted from chemical factors, which produces some direct role and can not produce the equivalent physical effects. So it must be equivalently combined with the physical therapy, then it could switch to a high-level treatment with direct action on the human body. Such a one-sided opinion that a certain drug, treatment device, gene therapy have a key role in curing cancer diseases is completely wrong, and must be treated using advanced treatment technology for the overall.

### (IV) Views of Knowledge Different from Introduction of Diseases in the Modern Medicine

The concept of disease is a general understanding of the nature of the disease, which thereby determines the principles of disease prevention and control measures. Modern medicine does not have a significant effect in treatment of major diseases, indicating the existence of its one-sided understanding of the disease. To cure major diseases must have the overall comprehensive understanding of the disease concept. For this, reference to the Chapter II “Introduction of the Disease”, Pathophysiology, 6^{th} Edition, and a textbook for the national institutions of higher learning.

(1) The concept of disease: modern medicine believes that disease formation is a process of the law on pathogenic damage and the body's anti-damage under certain conditions, of an injury resulting from the struggle with the anti-injury regular process, is the body under certain conditions, it's abnormal life process resulting from self—stability adjustment disorder due to pathogenic damage under some circumstances. The existence of both diseases mutually exclusive of the injury and the struggle of opposing anti-injury, and there is a mutual attraction function interdependent harmony with the recovery of identity, illness is the body against injury and structural damage, functional decline and recovery of the combined effect of the overall process of change, rather than a single injury and part of the process of struggle against injury.

(2) Causes of disease: modern medicine believes that the causes of diseases are specific factors to determine disease specificity. A disease can be caused by one or a variety of causes at the same time or one after another, so they must have specific analysis. The body own physical treatment of a variety of measures based on efficacy, physical decline is a universal risk factor. The causes of major diseases is a risk factor-specific internal structure and function of the universality of external pathogenic factors of the two causes of mutual constraints of the overall results of the combined effect of a disease does not cause serious illness, a variety of causes of non-existent.

(3) General rules for occurrence and development of diseases: (a) injury and anti-injury, modern medicine believes that the struggle between damage and anti-damage forms the basic driving force for development of diseases and the balance of power between them often influence the development trend and outcome of diseases. However, the specific forms and scales of fight against lesions are constrained by the identity of functional recovery and its role is limited. (b) The causal relationship, modern medicine believes that in the development of the disease, if the causal transformation and the vicious circle are interrupted, the disease can be beneficial towards the direction of the development of rehabilitation, which is the key for treatment of diseases. Stressing concrete, specific, and detectable factors and causalities while lacking the universal, general and undetectable factors and causalities will result in a temporary result easy to recur in treatment, which is causality between partial factors. Major diseases should be treated with the measures based on the specific link with the universal joint, which can really interrupt the overall transformation and the vicious circle and enable them towards the development and outcome beneficial to rehabilitation.

(4) Local and overall: modern medicine believes that all diseases are almost formed as a whole and the pathological changes in various parts are local performance of the systemic diseases, which take in the dominant position should be made a specific analysis. The theory used in the treatment of common diseases is correct, but in major diseases, the local lesions and overall disease are interdependent, such as a unified whole in the two components, there is no part of which dominated play a decisive role, can not be transformed into each other without a specific analysis, is another factor to promote the overall development of external linkages play a decisive role. Overall function of the major diseases and local disease refers to a structure adjacent to the level of overall function, not referring to the overall function of the body, in cancer cells means that the local structure and can not be separated from the overall function of the organizational system. Function does not occur as a result of systemic disease, does not exist between the interdependent relationship between the promotion of the whole, no matter what the level is not the location of the local structure of the cancer lesions, have to be a lack of hierarchy, the overall function of the body targeted therapy, and is to give up on the real occurrence of disease overall function of the key parts of treatment.

(5) Outcomes of diseases: Modern medicine believes that the outcome of patient rehabilitation and death depends largely on the balance between structural damage and struggle against damage. However, rehabilitation is very low cure rate and a large number of clinical facts have proven that: the majority of cancer patients die of the disease rather than changes in the structure, but the decrease in bodily functions. Metabolic function in the human body is the cause of death, and the balance of forces in the fight against anti-injury is aggravated or mitigated by conditions, to accelerate or delay this process, the restoration of function of disease treatment can stop the trend of development, not structural damage and death arising from the mutual constraints the overall effect.

### (V) Summary

(1) Basic principle: the overall therapy for major diseases is established from development of Chinese medicine theory on the basis of the guidance of modern philosophy and modern science and technology. It's significantly different from the approach to treat major diseases with modern science and technology. The overall pathogenesis treatment is a combination of the existing technology for anti-injury treatment and the novel technology for treatment of recovery of the overall combined, and the incidence at the target site, structural damage in the functional recovery, which unify to aim at the pathogenic sites and play a role in treatment of functional decline due to the overall mutual constraints within the whole quality changes, not for functional recovery of the whole body, because there is no overall functional lesions that need first aid. The common causes for occurrence of cardiovascular and cerebrovascular diseases and cancers are based on the overall functional lesion around local structures, involved in the inadequate supply of oxygen and nutrients metabolism, decline and damage of respiratory and circulatory functions, so the balance treatment of functional restoration becomes the critical factor for curing major diseases. It applies to cardiovascular and cerebrovascular diseases, diabetes, uremia, cancer, and AIDS and it can have a significant therapeutic effect.

At present, the overall awareness of the disease has not been taken as a basic contradiction involved in medical physiology, pathology and etiology and not as a general concept incorporated into clinical diagnosis and treatment. In modern medicine, the structure and function, specificity and universality, physics and chemistry, gradual changes and mutations appears not as a unified whole, but only concern about the structural damage and pursue overall efficacy through partial treatment, using primary technology to achieve high-level technical role. Such treatments mainly include chemotherapy, surgery, radiotherapy and other function-restoration less treatment. It will constrain and offset the role of anti-injury treatment. No matter how anti-injury treatment to enhance development and increase the cost of treatment, they can only play a limited role in part, does not produce significant overall effect, which is why the current cause of cancer diseases has been unclear, leading to the treatment of the direction of error, can not be reduced morbidity, mortality, there is no sense of the cost of cure may be the root cause of rising. Only basic medical theories of innovation and the adoption of the overall treatment of the etiology and treatment of high-level technology, diseases can be cured of cancer and reduce the cost of treatment.

(2) Random control test: The principles and methods of overall treatment significantly differ from modern medicine. As practice is a standard for test, employ practice to determine the correct principle and method of treatment. The overall treatment and modern medical treatment can also have the same test and the evidence to evaluate the efficacy comes from randomized controlled trials and a reflection of patient consensus.

The contrast of overall causal treatment and the existing technology: in the overall treatment, the functional recovery therapy should be carried out in the lesion site symmetrically and equivalently together with surgeries against damages, radiotherapy and local chemotherapy, through introduction of advanced treatment technologies to mutually improve efforts. And according to the overall treatment, identify treat in the trial group. The best technology available in the efficacy of anti-injury surgery, radiotherapy and chemotherapy treatment and support measures in the control group for treatment. Test Objectives: in patients with advanced cancer of the physical, structural change and the spread of the transfer of treatment, symptoms improved, the cure rate, mortality rate. Difference: not only the overall treatment of the completion of existing technologies in the treatment of anti-injury standards, but also to adopt a double standard on the completion of the restoration of function, such as standards, and anti-injury treatment of each other and have the overall effect, on the whole and parts, advanced technology and the primary technology significantly between the different nature, resulting in a significant treatment effect is different from the treatment of cancer diseases to determine the overall principle and the correctness of the method the only criterion is: Compared with the existing technology the nature of change and have a significant effect, to achieve the purpose of curing cancer disease.

### (VI) Causes of Cancer Formation and Development, Prevention and Treatment

The metabolism constitutes the basis of life existence and functional decline and imbalance is the cause of generation of cancer cells and carcinogenesis. The functional decline and disorder of the overall tissues around such cells is the external cause of damage to the DNA structure. When the cell internal structure can not adapt to external changes, it will develop into cancer cells to adapt to the role of external factors, which belongs to the metabolic diseases. Of the external conditions resulting in functional decline, smoking, unhealthy lifestyles and chronic diseases cause long-term function consumption and structural damage. Modern medicine has so far no conclusion on causes of cancer, which can only alleviate and eliminate the symptoms of structural damage but not prevent and treat cancers from the overall root cause. The overall unity of the human body indicates that all levels of the body itself have both specificity of development and universality of their mutual contacts. The upper level and the lower level constitute specific life events and the overall lesion through their interactions. The causes of cancer diseases lie in decline of external functions and damage to internal structures in cancer cells. Damage to the structure will also affect external functions. Their mutual constraints produce the overall nature of carcinogenesis, so it's necessary to treat the whole pathogenesis.

Cancers and major diseases develop from the common diseases where the functional decline is the cause of structural damage, inflammation variation and carcinogenesis as well as the cause of difficulty to cure. So as long as always conduct functional restoration on the body's sites easy to initiate cancers and the sites with common diseases unable to be cured with long-term treatment, the functional decline would not damage the structure and not form cancers under constraint with structural damage. Functional restoration can be easily conducted with the cancer treatment equipment, which may not only prevent cancers, but also promote healing common diseases. Pulmonary respiratory system, liver, gallbladder, intestines, stomach, pancreas digestive system diseases when under treatment for functional recovery still need the horizontal oxygen supply device to restore respiratory function, promote blood circulation and massage internal organs to recover digestive system. For details, refer to the instructions of the cardio- and cerebrovascular disease treatment equipment (the patent application number No. 200610069553.8.).

It's reported from the Chinese medicine exclusive column on the 3^{rd} page of the Health Newspaper on Jul. 20, 2006 that in the early 1990s, a Canadian oncologist, Professor Schipper believed that overly killing cancer cells would damage the normal reactions of the body to deteriorate and disorder imbalance of antibody-intermediated regulatory functions. Effective treatment does not require complete tumor regression and kill all the cancer cells, but to improve their genetic status and the ability to self-rehabilitation. It's reported that the Secretary of Science and Education Division, the State Administration of Traditional Chinese Medicine, Professor GAO Sihua said: “Chinese medicine wins in the treatment of cancer in a way of ‘orderly treatment’ and ‘holistic treatment’”. Different from treatment of cardio- and cerebrovascular disease, in cancer treatment, functional restoration, deepening the level of treatment against structural damage and specific location are difficult for doctors and patients to grasp, it is the reason for difficulty in prevention and treatment. However, their principles of supporting healthy trend and removing bad aspects are consistent. To kill cancer cells can not recover with the restoration of function with the same need to kill cancer cells, cancer treatment is a problem, such as the composition of the two together can not be separated, there is no relationship between the main secondary. Existing technologies in cancer treatment depend on the restoration of function is secondary to those who assisted in the indirect effects from systemic functional recovery, injury treatment with anti-promote each other on an equal level of high-level nature of change and significant efficacy of cancer treatment actually declined in a restraint by functional role of anti-injury treatment was offset by the state.

In the overall treatment, the effort of anti-damage treatment of killing cancer cells and the scope reduce and retract into the field of equivalent treatment as to exit from the present dominant position of cancer treatment and to abandon the goal using radical surgery to remove the tumor and radiotherapy and chemotherapy to kill all cancer cells, but to ensure partial and limited anti-damage treatment on the condition of restoration of function. Through supporting healthy aspects and removing bad aspects, unify their effects to treat cancer. Recovery through the use of specific targeted therapy and advanced treatment techniques such as access to part of the status and scope, yet the current support, secondary position. The restoration of function and anti-injury of their own development as a combination of both, such as the overall treatment, treatment and chemical treatment can not afford their own separate development, the combination of the two sides on an equal treatment as a senior.

Because cancers at an early stage are of small-scale and low-level, excision will not significantly reduce the surrounding functions. The function of restoration can restrain proliferation, development and transfer of cancer cells that can be surgically removed, and the functions of the structure around the focus can also be resumed. As long as one side's factor is normal, the proliferation of cancer cells will not transfer into the intermediate and advanced stages, maintaining in a long-term coexistence with the normal body. The two sides are in a dominant and subordinate relationship and the dominant factor play a decisive role which can be cured by existing treatment methods. Intermediate and advanced cancers should be subject to the holistic treatment and advanced technology. Only in this way can the cure for cancers be resulted, because the functional restoration can eliminate the damage from surgery, radiotherapy and chemotherapy and ensure normal anti-injury treatment, besides it can eliminate the basis for recurrence of cancer cells so that the development direction and trend of cancer begins reverse, significantly reducing the patient mortality. It's the whole treatment and introduction of advanced treatment techniques that achieve the purpose of curing cancer, rather than relying on the current dominant treatment killing cancer cells and adjuvant systemic therapy.

### (VII) Prevention and Treatment of Cancer Progression and Metastasis

Changes in the overall functions can constrain normal cells, promoting them to transform into cancer cells and it may also constrain cancer cells, promoting them into normal cells. The decline in function of normal cells and the structural damage caused by cancer cell proliferation are the two symmetrical components of the whole cancer disease. Lack of one component would lead to no existence of the other as they can't be classified which is dominant or subordinate. Suppression of proliferation of cancer cells doesn't mean growth in the number of the surrounding normal cells. The whole living surroundings of cancer cells is an anoxic and ischemic microcirculation disorder where the interaction between cancer cells and their adjacent surroundings' functions is the key to determine whether metastasis occurs and metastatic sites. According to the “New Ideas on Treatment of Cancers” on the page 36, vol. 5 in 1997, Grand Garden of Science, said: “Hoffman et al from Harvard Medical School believe that excision of cancer cells with their functions constrained in silent period often does not cure the cancer, but can promote its explosive growth, as well as the risk of proliferation and metastasis. This is because anti-transfer components in patients immediately disappear along and the balance is broken, so the occurrence of metastasis is also difficult to avoid”. Gene therapy and resumption of the whole gene function of the adjacent structures can constitute a mechanism to eliminate causes and treat cancer cells; however, the main threat to life is the proliferation and metastasis of cancer cells. Therefore the main current tasks to suppress proliferation and metastasis of cancer cells are to kill cancer cells and to recover tissue, organ and system levels of functions.

Cancer cells are relatively large and not suitable for circulation, so they are less likely to be lingering in the body. The systemic chemotherapy is thus limited in its efficacy against proliferation. The metastatic direction and site of cancer depends on pathogenesis at the anatomical location of the structure, lymphatic and blood transmission, and the functional decline of the surroundings, the environment suitable for survival and development of cancer cells. As said from an article of the New York Times on August 15, published on Reference News on Aug. 28, 2006, “the environment of the tissue attacked—microenvironment, playing a key role in metastasis of cancer cells”. Such argument identified the function of microenvironment, but with one-sidedness from the previous emphasis on proliferation of cancer cells to on functions. Cancer spread and metastasis is the whole result of interaction between proliferation of cancer cells and functional decline in the environment. To kill cancer cells alone or only to rely on restore the micro-environment can not stop the spread and metastasis of cancer. The key point to stop the spread and metastasis of cancer cells is the holistic treatment using the anti-injury treatment to kill cancer cells and to restore function of micro-environment and equivalently introducing physical and chemical energy into normal cells. Such treatment will suppress metastasis of cancer cells. Even if not prevent the cancer metastasis, it can exercise a curing function.

Although the existing technologies such surgery, radiotherapy and chemotherapy are applied to the anti-injury treatment and can temporarily alleviate symptoms, they can't recover or improve the situation of decline in the whole functions of the structures around the focus and decrease in the normal cells. On the contrary, a new blow occurs and destroys the whole functions around the focus that may recover. Because the functions around the focus of patients at intermediate and advanced stage are at a high level of organs and the system, such a new blow would be fatal and it is a treatment at the expense of basic long-term survival functions, just maintaining the structure. Through such treatments, patients lose the survival function and the trend to die has been determined, which is no more than surviving time maintained. Different acute infectious diseases, implanting cancer cells in laboratory mice that mainly respond to the external factors and can quickly be cured, cancer and cardio-cerebral vascular diseases are chronic diseases, caused by energy consumption and lack of supplying structure damaged, so they can neither rapidly cured, nor promptly cause death. Performing an injury treatment to kill cancer on the basis of significant decline in the whole functions, short-time radical surgery is at the cost of life and there is no possibility of success. It's reported from Science and Technology Daily on Dec. 1, 1997 that according to Japanese scientists' research on patients under radiotherapy and chemotherapy, the possibility of metastasis and spread of those patients is 8 times as much as the patients without such treatments. As reported by the Peninsula City News on Jan. 16, 2003, the metastasis and recurrence proportion of tumor after surgery, radiotherapy and chemotherapy was 69% and 90% within one year. 50% of the patients died in the factors during surgery, radiotherapy and chemotherapy. A large number of clinical facts show that the direction reason for majority of cancer patients are not cancer cells themselves, but the decline in their functions and bacterial infection. 90% of patients died of cancer spread and metastasis. If patients have the metabolic function, it would not result in the external environment suitable for the metastasis of cancer cells, and not cause to die with interacting with cancer cell proliferation. Patients, although seriously ill, will not die. This is why patients with intermediate and advanced cancers can't survive for 3 to 5 years cancer. The advanced technology combining physics and chemistry can not only restore function, perform the overall anti-injury treatment and achieve the purpose of prevention and treatment of cancer spread and metastasis, but also reduce the treatment time significantly than the existing technology.

### (VIII) Functions of Cancer Treatment Equipment

The human body functions are dependent on mechanical energy, heat, electricity, chemical energy. Cancer is a pathogenesis with partial structural damage and functional decline. Around the cancer lesions shortcomings and obstacles occur to the functions of thermal energy maintain body temperature, the mechanical energy for muscle contraction, electricity for nerve impulse conduction and the metabolism of supply and absorption, and blood microcirculation, they are holistically in a downward trend. It's the basis for normal cells transforming into cancer cells. The mechanism of the cancer treatment equipment is that since the mechanics is the foundation of sound, light, electricity and magnetism, combining mechanical massage, ultrasound, electrotherapy, magnetic therapy, light therapy and physical thermal therapy into a holistic treatment in order to significantly improve the physical effect, on an equal footing with the primary chemical form of the overall treatment of local transdermal delivery, local injection, oral administration and intravenous administration that can be incorporated into a high-level treatment, around the patients' lesions input mechanical energy, thermal energy, electrical energy, sound energy, magnetic energy, chemical energy, etc., significantly increase the overall the degree of functional recovery for the causes. The cancer treatment equipment can kill cancer cells by controlling the physical reaction intensity and use of anti-cancer drug. The chemical substances used can be divided into that for recovery of functions such as vitamins and nutritional medicines as well as antibiotics and anti-cancer drugs. Existing ways of oral drug delivery and systemic intravenous administration is less targeted, belonging to the systemic delivery. Cancers should be treated through local administration as much as possible.

In the treatment to restore the overall functions, it should be carried out with surgery, radiotherapy and chemotherapy in a symptomatic and equivalent way so that the overall functions can be protected and restored, increasing the treatment sensitivity. In such way, cancer cells don't have the opportunity to spread and the effect of surgery, radiotherapy and chemotherapy would be improved to bring development trend of the disease under control. The purpose of symptomatic treatment of cancer turns to the local physical treatment such as surgery and radiotherapy, however, the equivalent treatments such as the local transdermal delivery and drug injection are considered subordinate. These primary treatment does not equivalent incorporate the physical and chemical treatment and results in limited efficacy. With the cancer treatment equipment, both the aspects can be symmetrically and equivalently combined into a high-level treatment and produce a significant effect. The treatment factors and features of the cancer treatment equipment are as follows:

(1) Mechanical massage: elimination of obstacles to the blood microcirculation is the key to restore functions as well as the main reason for surgery, radiotherapy and chemotherapy can't cure cancers. However, elimination of microcirculation obstacles can not be separated from mechanical massage and drug massage. The driving forces for microcirculation come from the mechanical movement of the surrounding tissues, so simple heating, electrical therapy, drugs and antihypertensive are limited in their effects. Local microcirculation has nothing to do with increase of the systematic blood stream and blood pressure changes because when blood pressure changes, capillary veins have no discernible changes in diameter. 99% of their stiffness forms by the surrounding tissues. Mechanically, it should take the capillary veins and the surrounding tissues as a whole. So only through drug massage and mechanical massage to make stiff muscles relaxed and through heating and delivery of drugs, the capillary veins can be smooth, blood flow can be increased by 10 times and the microcirculation obstacles can also be eliminated. Since the mechanical movement forms the foundation of mutual transformation of the thermal energy, electrical energy and chemical energy, the existing technology lacks mechanical movement and other therapeutic treatment is carried out in separate at a low level, rather than a high-level holistic treatment technology combining mechanical, physical and chemical energies. So they become the reasons for the low efficacy.

Massage and heat alone, it is to promote the transfer of the proliferation of cancer cells may be because the transfer of the overall impact of the proliferation is a response to the treatment of the role of some factors that will promote accelerated this reaction. Cancer treatment is the treatment of the overall high-level technology, contain anti-cancer drugs, the restoration of function in tumor cell killing at the same time to eliminate the proliferation of factors, a separate application for pain relief, prevention of proliferation, improve physical fitness. Existing technologies such as ultrasound therapy in the treatment of tumors is a taboo, but the use of ultrasonic transdermal anti-cancer drugs enter the tumor before treatment, except table. Massage therapy is so taboo, but the importation of anti-cancer drug Massage injection drugs and anti-cancer drugs with the exception of the treatment of tumors. Treatment of advanced cancer in the period as an integral part of the overall treatment, radiotherapy and chemotherapy and surgery at the same time part of and promote each other through the whole treatment of the role.

Massage can make the number of capillaries per square millimeter open from 31 to 1400, speed up the lymph flow by 7 times. The friction head is divided into two types: one is squeeze-type head and the other is static friction head, driven by the micro-motor, speeds at 300 rpm, 1000 rpm and 1800 rpm. The work angle of the squeeze-type friction head is acute, similar to the cutting and extrusion role of drill and its end is in the form of half-moon, friction heat fast, high heat production, suitable for the role of the skin surface and the molecular weight of 1000 through the following drugs Transdermal drug delivery treatment. Because of human skin stratum corneum is the key to both transdermal level, with the stratum corneum was in the forms of scale, acute angle of friction can be generated when the first rotating squeeze into the role of thermal expansion of the skin to be squeezed through the stratum corneum drug molecules into the subcutaneous treatment of the role of organizations. And existing technology in sliding friction is the obtuse angle of the first work is the wave-shaped reinforced wheel protruding friction head, vertical, rolling when the role of rotation can not be administered through the stratum corneum in the forms of scale can only increase through the vertical pressure to make the drug through the sweat glands, pores into the subcutaneous tissue of the body, dosage restrictions, and cut into the extrusion-type friction effect of the first transdermal drug delivery, there was a significant difference. The friction head for deep tissue muscle massage, it pressed cloth and skin drugs, and of movement between the skin to stimulate skin muscle kneading, subcutaneous deep tissue, resulting in endogenous heat, relax muscles, and matched by the promotion of local drug injection Drug absorb blood circulation. Of two friction devices in the treatment of head can be installed at the same time working together, can also be installed separately to separate the work of skin friction between the permanent drug cloth, the friction head pressure 0.25 kg-0.5 kg per square centimeter kilograms on the skin and points and when pressuring 0.5 kg-2 kg per square centimeter, it may reach the deep muscle layer. When the 3-5 kg it may reach deep tissue and bone surface, the drug absorption depth through drug massage is the same as the mechanical massage and electrical stimulation.

(2) Treatment of cancer of the mechanical massage device, drug Massage, DC, LMF electrotherapy, magnetic therapy combined nerve and muscle can be treated lesions, because most of the muscles in both the distribution of a large number of nerves, is often difficult to muscle movement and nerve stimulation separately, muscle movement is inseparable from the nerve stimulation, there are a whole between the two inevitable, such as the ischemia resulting in muscle stiffness is not going to stimulate nerve conduction obstacle to muscles, blood circulation, oxygen supply. Lesion in cancer patients, the nerve and muscle diseases have occurred, both the mutual constraints of the overall decline in physical function component of the reasons, the treatment by a single factor in the effect of symmetry factors is limited, the need for mechanical energy, electrical energy between the promotion of the overall treatment, can play a significant effect. Squeezed into the device in the treatment of friction static friction head and the first micro-motor driven by the rotation of the skin friction and the middle of the first drug of drug coated cloth, wire DC pulsed metal extrusion and friction into the first connection, direct current pulse through a metal friction head direct treatment, drug treatment combination of massage with the DC as a whole. Low-frequency, medium frequency electrotherapy electrode plate made of semi-circular eccentric head-like protrusions of static friction, when the static friction will be the first drug is relatively compact suction skin, the low, medium frequency electrotherapy and massage the muscle kneaded into the overall treatment At the same time, static friction can also be made of metal head shape, with the combination of DC pulse. Magneto therapy role: in the metal into the extrusion head and low friction, medium frequency pulse current of the foundation posted a magnetic disk can be on electrotherapy, and therapeutic effect will be even better.

(3) The treatment of cancer diseases in the mechanical massage devices and radio frequency diathermy, microwave heating of ultrasonic heating the combination of existing technologies, microwave heating hot but the lack of regulation is often easy to form the phenomenon of heat pent-up, limiting effect of play, the need to massage the promotion of with blood circulation. Ultrasonic heating and massage combine to eliminate the air organ fasting and other restrictions, low-level tissues in the muscles, nerve function, the ultrasonic treatment of the role of depth and heat production will be to promote improved, while promoting a deeper level of confrontation cancer drugs, nutrition absorption and utilization of drugs. Treatment of the direct use of existing radio frequency technology, microwaves, ultrasound treatment equipment, treatment of disease and cancer combined treatment devices.

(4) Cancer treatment device in the administration of mechanical massage and vacuum combination of human skin stratum corneum is in the forms of scale with vigorous resistance to mechanical compression force, organization of drug molecules to enter the skin through the stratum corneum, and negative pressure so that it can improve the anti-Zhang rank structure, so that the effect of friction to increase the number of extrusion times. Under negative pressure, the skin can increase the elongation rate of 40 percent less than the skin from the damage, with the heat expansion of the stratum corneum to increase space, pores, sweat glands had a diameter of the corresponding expansion of friction for transdermal delivery of macromolecules anti-cancer drugs, nutrients through the cuticle to create the conditions to enter the subcutaneous tissue. Treatment is low, medium frequency pulse current and the negative pressure combined with transdermal delivery of equipment for treatment at the same time, negative pressure suction can be used to generate electricity, but also can pinch. It has the role of negative pressure therapy have a rubber ball This abdominal pressure on the human body have a similar role in Traditional Chinese Medicine Cupping treatment, negative pressure existing products are rubber balls.

(5) Combination of the mechanical massage and drug therapy of the cancer treatment equipment. In cancer therapy, local injection and local transdermal delivery is an irreplaceable chemotherapy equivalent to surgery, and physical radiation therapy. However, the existing technologies utilize such oral administration, intravenous systemic administration that can't play a key role to replace local administration treatment. When the overall function of local cancer declines, such administration just make drugs accumulated around the tumor and they can not circulate into the tumor through the blood vessels, resulting in only one-tenth of drug utilization. Drug delivery and absorption is a critical component of chemotherapy, which needs local drug delivery and transdermal delivery. In a way appropriate to transdermal delivery for low level tissues, external drug, no matter which is cream, liquid cream or oil, must take effect through in-vitro massage. The existing physical penetration technologies include iontophoresis, electroporation, ultrasonic drug delivery and skin penetrating agent, all of which are single penetration promoting factors, lacking mechanical massage extrusion and friction heat treatment and therefore the effect is limited as the drugs with molecular weight of more than 1,000 can not enter the skin. Such approaches are not popular so far. And transdermal delivery through massage, named as the cream massage in ancient times, was widely used in Han Dynasty of 2,000 years ago. According to Chinese medicine theory on the cream massage and friction chemical technology, the treatment equipment makes a transdermal friction delivery through mechanical actions. The temperature of the skin massaged may rise up to 45-50□ at 1800 rpm for 10 seconds. Maintain 6 minutes to soften the stratum corneum and the gap increased; meanwhile the extrusion force drives the drug molecules into the subcutaneous tissue through the stratum corneum. The static friction head also work to make muscles heated internally and to promote blood circulation and absorption of drugs. The department of biomedical physics, University of Munich, Germany, developed a new transdermal delivery vector—transfersomes. Such vectors can be deformed through the aperture is one-fifth of the skin's own channel and its integrity is not affected, so penetration of macromolecular drugs into tissues to carry out the deep systemic administration becomes possible. For details on preparation of transfersomes, see New Transdermal Delivery Mode (1^{st} edition in December 1997), ZHENG Junmin, People's Health Press; Development and Prospect of Pharmacy (1^{st} edition in July 2007 (page 38-39)), WU Lei, PING Qi-neng, Modern Biotechnology and Medicine Publication Center, Chemical Industry Press; The lipid part of transfersome is composed of 20-50 mol % (10-24 w %) of phosphatidylcholine, an appropriate amount of sodium cholate and 3%-7% of ethanol or propylene glycol. The physical friction transdermal delivery of the treatment equipment will produce a significant effect with such transfersomes.

(6) Combination of the low-level and high-level treatment. For tumors at a low-level site, it should use transdermal friction administration. The static friction make muscles “heated internally” to promote blood microcirculation, which combines DC, low-frequency electrotherapy, heating, vacuum rubber balls, magnetic therapy, anti-cancer drugs 5—fluorouracil, nutrients such as vitamins C and D3, thermogenesis and blood flow promotion drugs and carriers for transdermal drug delivery—transfersomes to form a holistic treatment, eliminating local damage of surgery, radiotherapy and chemotherapy and restoring the overall function at the pathogenic sites. Through addition of appropriate anti-cancer drugs, it may prevent proliferation of cancer cells. Local injection and intermediating administration at enhanced dose may directly treat cancers with functional restoration treatment; meanwhile the intensity of mechanical massage and ultrasound heating temperature of electrotherapeutics should be enhanced.

For those tumors deep in more than 5 cm, when conducting low-level functional restoration, the combination of radio frequency, microwave, ultrasound, local drug injection and intervention administration methods can increase the intensity of functional restoration of the deep tissues. When conducting drug intermediated is treatment and local injection, inject with a syringe at an oblique angle. The pinhole is enclosed with adhesive tapes without an obstacle to the work of the treatment equipment. It can be used in a way of needle-free injection, arterial injection, intrathecal injection and injection of tumor. When simultaneously using functional recovery both at a low level and at high level, it will make an overall significant effect. Due to lack of promotion of low-level action, the high-level action is also constrained. Because the intensity of physical therapy increases, the deep tumor sites may be directly injected with nutritional medicine, anti-cancer drugs into the local blood vessels and the surrounding points and pain points. By promoting blood circulation for absorption of drugs and ensuring the restoration of function, on this basis it may employ anti-cancer drugs in treatment of cancers. It has been clinically proven abroad that when a patient body temperature rises to 42 □, it should use anti-cancer drugs such as adriamycin, bleomycin and nitrosourea with the efficacy 10-100 folds than that at the body temperature of 37 □

(7) Combination of functional restoration and anti-injury treatment to kill cancer cells. According to the causal relationship, Spread and transfer of intermediate and advanced cancers develop as a result of the internal cause of cancer cell proliferation and the external cause of decline in functions of the surrounding structures. Killing cancer cells alone just produces partial causality instead of the overall effect. So it must take measures to form a holistic treatment that can inhibit proliferation of cancer cells and restore the functions of the surrounding structures. In the treatment of advanced cancer, the cancer treatment equipment can be cooperated with surgery, radiotherapy and local chemotherapy. For prevention of the spread and transfer of cancer cells, it can be used separately. Other configurations can reach the purpose of this invention.

### BRIEF DESCRIPTION OF THE DRAWINGS

FIG. 1 is the front view of the cancer treatment equipment under this present invention.

FIG. 2 is the view of the squeeze-type friction head of the treatment equipment shown in FIG. 1.

FIG. 3 is the view of the static friction head of the treatment equipment shown in FIG. 1.

FIG. 4 is the view of the pathogenic sites and positioning treatment for major diseases.

FIG. 5 is the schematic diagram of overall high-level treatment.

### DETAILED DESCRIPTION OF THE INVENTION

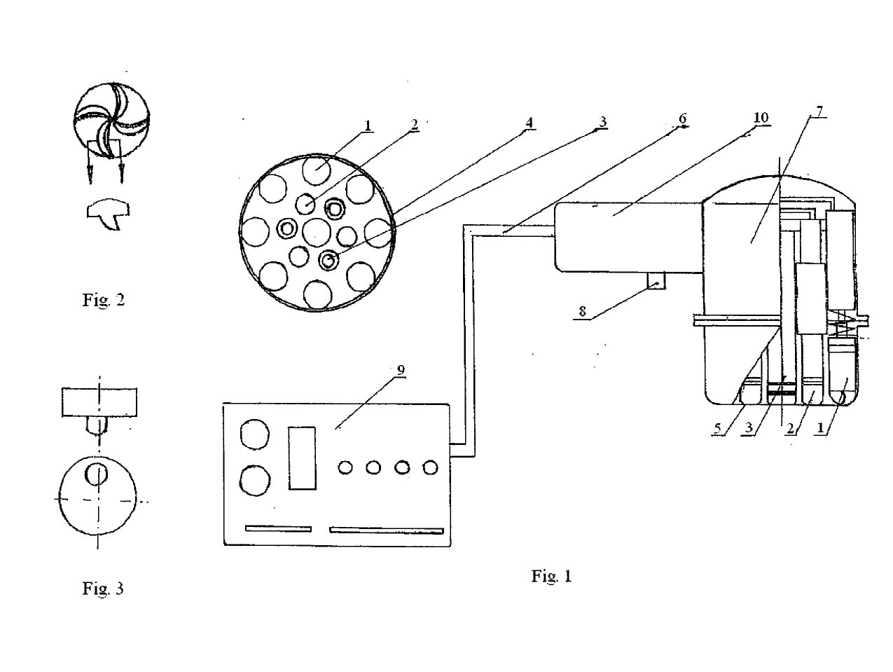

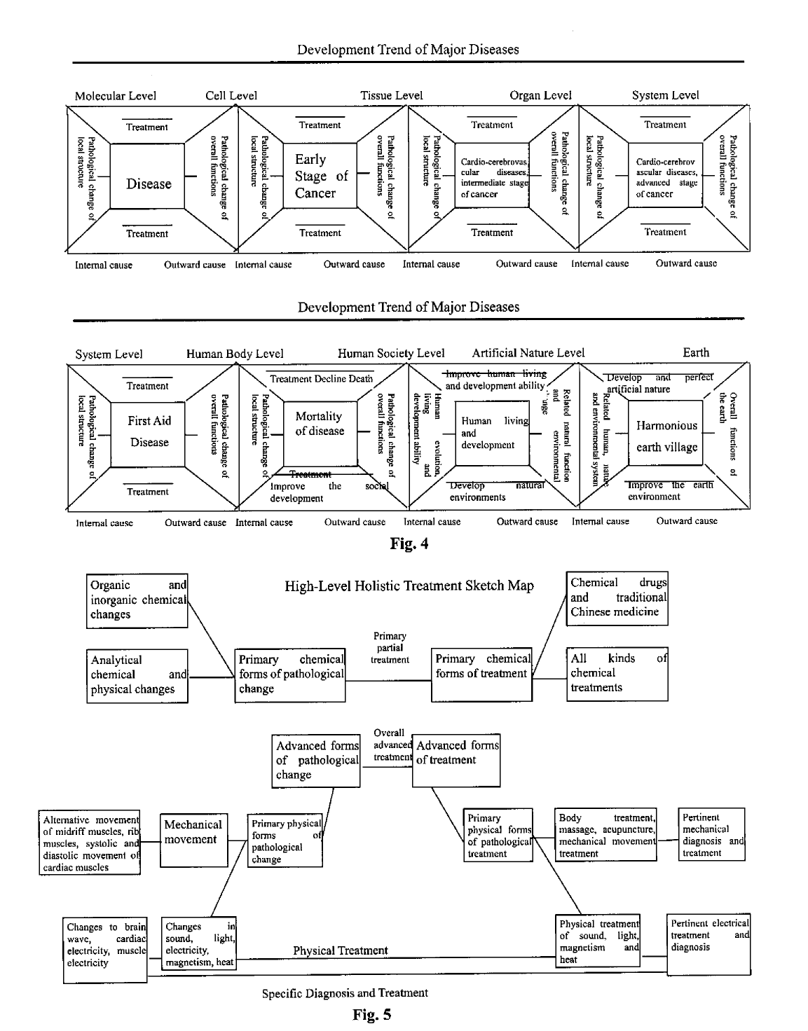

As shown in the FIG. 1, the invented cancer treatment equipment is composed of 1, DC squeeze-type friction head; 2, LMF static friction head; 3, friction head with a magnetic sheet; 4, medicine gauge fixed pad; 5, medicine pad; 6, cable duct; 7, case; 8, switch; 9, controller and 10, hand. FIG. 2 is a view of DC squeeze-type friction head. FIG. 3 is a view of LMF friction head.

The invention of the cancer treat equipment is achieved through the following technical measures: 1, DC squeeze-type friction head; 2, LMF static friction head; 3, friction head with a magnetic sheet; which are in parallel installed to inside of the fixed case 7. Their ends locate at the same horizontal level. The squeeze-type friction head 1 is located within an external circular of the case 7 inside which there are interlaced with the static friction head 2 and the friction head with a magnetic sheet 3. In the center of the shell 7 is also located with the squeeze-type friction head 1, each equipped with a micro-motor. Each friction head are respectively connected DC and AC power through the cable duct 6, grasp the hand 10 to switch on the switch 8 to start work. Under the case 7 there is the medicine gauze fixed pad 4 on which there is the medicine pad 5 with drug solution. The controller 9 is used to control DC, AC power and voltage and produce pulsed current and output current. The work angle of the squeeze-type friction head is acute, in the form of half-moon convex slope and in the shape of characters “□” and “□” or “□” in row. They are made of metallic materials and abrasion and corrosion-resistant rigid plastic material. The static friction head 2, shaped in a semi-circular convex, can press and suck the medicine pad 5 as well as rotate the skin. The friction head electrode is made of metal, conductive rubber and conductive cloth material. The conductive rubber is molded into the work-type friction head where the friction head with a magnetic sheet 3 is fixed. The RF, microwave, ultrasonic treatment devices and negative pressure rubber ball of the cancer treatment equipment are products by existing technologies, which can be directly purchased for joint treatment.

The working principle of the present invention: (1) the squeeze-type friction head of the cancer treatment equipment is suitable for skin friction and heat production, transdermal drug delivery; DC is suitable for expansion of blood vessels, promoting blood circulation and transdermal delivery, and promoting metabolism of local tissues and antimicrobial action. The mechanical actions, although different from DC treatment, is the same as in the level of treatment sites and purposes. The body requires actions of both sides which can be unified to significantly enhance the intensity. The DC pulsed cable of the cancer treatment equipment is used to link the squeeze-type metal friction head for joint action.

(2) The static friction head is suitable for massaging deep tissues and muscles massage to promote absorption and utilization of local drug injection. The LMF pulsed current is mainly used to excite nerves—the muscle tissue will be accordingly caused contraction. Additionally, it can release pains and promote blood circulation. The LMF pulsed current therapeutic plays the same role as the LF does, but it can enter deeper subcutaneous tissue have better efficacy. Home treatment devices are mostly of the type to produce LF and MF. Kneading massage and LMF electrotherapy can restore muscles' nerve functions in decline from two different aspects and have a significant effect. The electrode plates used in the LF and MF treatments are made into a semi-circular convex static friction head, which is connected to the cable to holistically make the LMF electrotherapy stimulate the nerve and use static friction massage to relax the muscles. At the same time, the static friction head can also be made of metal head shape, used with the pulsed DC.

(3) The RF, microwave, ultrasonic treatment devices of the cancer treatment equipment are existing products, which are essential in cancer treatment, if absent, it's unable to conduct deep-level and overall treatments, and thus the cancer treatment has become incomplete.

(4) In the treatment equipment where the mechanical physical therapy, local transdermal delivery, local injection of chemical drug combine as a whole into an advanced treatment technology for local function restoration. Together with the existing treatment technologies such as surgeries and radiotherapy treatment against damage, it can improve treatment of cancers.

Specific treatment steps are as follows:

1. When carrying out local low-level therapy, spread the nutrition drugs of vitamin D3, vitamin C, blood promoting circulation drugs and anti-cancer drugs on the treatment sites or the medicine pad. For high-level treatment, use local drug injection, drug intervention and microwave, RF, ultrasonic treatment.

2. Switch on the treatment equipment: at first, perform the negative-pressure operation of the rubber ball in the low-level treatment, at the same time carry out DC electrotherapeutics, transdermal massage delivery and magnetic therapy. Each site is treated for 3-5 minutes, and then followed by static friction massage to relax muscles. The LF electrotherapeutics is used to stimulate the nerves and heat. Drugs are administrated through local injection with each site treated for 3-5 minutes. It also can be treated simultaneously. High-level local injection, interference and radio frequency, microwave, ultrasound therapy can be combined with each other at the same time and the treatment time is 10-15 minutes. It may adjust strength of different factors according to the need for treatment. The restoration of function belongs to adaptation stimulation and should be subject to the comfort feeling of patients. Patients with lung cancer and digestive cancer should use horizontal oxygen absorption in treatment to improve the respiratory function and digestive function.

3. Once a day, each treatment program includes 15 times. It can be alternately used with surgery, radiotherapy, local administration of anti-cancer drugs. Such two aspects are combined and can not be interrupted, which can be performed before and after surgery, radiotherapy and chemotherapy at interval of 2-5 minutes, depending on the treatment situation.

4. Through speeding up rotation of the friction head, increase local transdermal delivery, injection dosage and strengthening depth of muscle tissue kneaded and the intensity of low-frequency electrotherapy, radio frequency, microwave, and ultrasonic treatment stimulating and heating the nerves, giving priority to using of anti-cancer drugs, the cancer treatment equipment alone can treat cancer diseases by killing cancer cells. The anti-injury treatment belongs to the non-adaptive stimulation□The patient does not feel comfortable.

5. The treatment to restore the functions in pathogenic sites is suitable for prevention, treatment of various tumors. For those weak patients losing the opportunity of surgery and radiation therapy, but can not use large-dose chemotherapy, it may have surgeries and radiation therapy and chemotherapy following the functional restoration treatment. Systemic adjuvant treatment such as oral medication, vein drop injection, nutrients and physical activity should be determined according to patient situation.

The example for application of the present invention: through mechanical massage of the cancer treatment device, use 4 “□” type, wave-shaped convex tendon sliding friction heads, each 1.5 cm in diameter, with speed control switch, to fix the skin protected by the friction medicine pad, which is an invention with the patent number ZL98110305.7. When the friction head imposes 0.5 kg of pressure per square centimeter and rotates at 1,500 rpm, it can produce heat of 50□ within 6 seconds to transfer the heat into the body through the pad. When the friction head imposes 3 kg of pressure and rotates 500 rpm, the massage force can reach the deep up to the deep muscles. The product has been audited by the Food and Drug Administration, Shandong, and awarded the document (LYG (W) 2000) No. 2020044. It has been used in Qingdao Tumor Hospital doctors for treatment of sites adjacent to the tumor focus and points with the treatment time set at 20 minutes, once a day and 15 times as a course of treatment. The majority of patients suffer from constitution deterioration, nausea, hair loss, and loss of appetite, pain due to surgery, radiotherapy and chemotherapy. Through the massage therapy with the treatment equipment, those patients are released from pains, and improve their quality of life and more they are likely to accept radiochemical treatment with better effect. Patients are generally willing to accept and hope implementation of self-operation. Doctors made a suggestion on further improvement and development.

1. A cancer treatment, using physical therapy and chemotherapy for patients with the restoration of function and anti-injury treatment on and around the focus site; it is composed of a DC squeeze-type friction head, LMF friction head, a friction head with a magnetic sheet, a medicated gauze pad fixation clamp, a medicated gauze pad, cable duct, case, switches, controllers, handles, and a device of RF, microwave, ultrasonic treatment and a negative pressure rubber ball; its characteristics are: the DC squeeze-type friction head (1), the LMF static friction head (2) and the magnetic-sheet friction head (3) is fixed in parallel to inside the case (7), and their ends are laid at the same level, DC wires are linked to the squeeze-type metal friction head that separately lies in the outer ring and the center of the case (7), the semi-circular-shaped raised static friction head (2) made of LMF electrode plate is interwoven with the magnetic-sheet friction head (3), each friction head has a motor connected to DC and AC hands (10) with cables (6), which is powered into operation with start switch (8); under the case (7) are there a medicated gauze pad fixation clamp (4) and a medicated gauze pad (5) smeared with the drug solution, the controller 9 is used to control DC, AC power and voltage, responsible for occurrence of pulse current and current output; DC squeeze-type friction head (1) has cutting and acute compression angle in form of curved half-moon inclined convex, in the character shape of “—” and “□” or “+”, the LMF friction head (2) is made of conductive rubber, the magnetic-sheet friction head (3) is fixed on the LMF friction head.

2. The cancer treatment equipment according to claim 1, being characterized in that the DC squeeze-type friction head is made of metal materials and corrosion-resistant□rub-resistant rigid plastic.

3. The cancer treatment equipment according to claim 1, being characterized in that the static friction head which can be connected to the pulsed DC power.
